(19) United States
(12) Patent Application Publication
(10) Pub. No.: US 2005/0124962 A1
(43) Pub. Date: Jun. 9, 2005

(54) Stretchable absorbent article including a suspended absorbent body

(75) Inventor: Cynthia L. Wyngaard, Kaukauna, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/729,485

(22) Filed: Dec. 5, 2003

(51) Int. Cl. A61F 13/15

(52) U.S. Cl. 604/391

(57) ABSTRACT

A stretchable absorbent article having an outercover, a bodyside liner joined to the outercover, and an absorbent body between the outercover and the bodyside liner. The absorbent article also includes at least one suspension member attached to the absorbent body in one of the waist regions and sandwiched between the outercover and the liner.

### FIELD OF THE INVENTION

The present invention relates generally to absorbent articles intended for personal wear, and more particularly to such absorbent articles having an absorbent body that is suspended between a stretchable outercover and a stretchable bodyside liner.

### BACKGROUND OF THE INVENTION

Absorbent articles such as diapers, training pants, incontinence garments, and the like conventionally include a liquid permeable body-facing liner, a liquid impermeable outercover, and an absorbent core (also referred to as an absorbent body) formed separate from the outercover and liner and disposed therebetween for taking in and retaining liquid (e.g., urine) exuded by the wearer.

In some of these absorbent articles, the outercover and/or the liner are stretchable to permit some expansion of the article when necessary to provide a better fit on the wearer. During use, the article is subjected to forces such as those generated by loading of the absorbent article and movement of the wearer. These forces can cause the absorbent body to shift within the absorbent article, to tear, or to otherwise become permanently distorted, all of which reduce the intended absorbent characteristics of the absorbent core and increase the possibility of liquid body exudates leaking from the article.

To this end, it is known to secure a substantial portion of the absorbent body to the outercover and/or the liner, such as by adhesive, thermal bonding or ultrasonic bonding, to inhibit the absorbent body from shifting as the article is subjected to various forces during use. However, securing the absorbent body to the outercover and/or liner tends to reduce the stretchability of the substrate to which the absorbent body is secured, and can impart a measure of inflexibility to the article in the area of securement.

There is need, therefore, to sufficiently secure the absorbent body within the stretchable absorbent article to inhibit permanent distortion of the absorbent body, while maintaining the stretchability of the stretchable components layers that, in part, make up the article.

### SUMMARY OF THE INVENTION

The present invention relates to a distinctive disposable absorbent article. In one aspect, the article defines a front waist region, a back waist region, a crotch region that extends between and connects the front and back waist regions, a longitudinal direction and a lateral direction, an inner surface and an outer surface opposite said inner surface, and a pair of longitudinally opposed end margins and a pair of laterally opposed side margins. The article includes a stretchable outercover, a stretchable bodyside liner joined to the stretchable outercover in a superposed relation. The outercover and the bodyside liner are joined along at least a portion of the end margins to provide a front waist seam and a back waist seam. The outercover and the bodyside liner are also joined along at least a portion of the side margins in each of the front waist region and the back waist region to provide a pair of side seams in each of the front waist region and the back waist region. In addition, the article includes an absorbent body disposed between the outercover and the bodyside liner. The absorbent body defines a pair of longitudinally opposed absorbent end edges and a pair of laterally opposed absorbent side edges. Further, the article includes a first elastomeric suspension member attached to the absorbent body in the front waist region. Still further, the article includes a second elastomeric suspension member attached to the absorbent body in the back waist region. The first elastomeric suspension member is sandwiched between the outercover and the bodyside liner in the pair of side seams in the front waist region. The second suspension member is sandwiched between the outercover and the bodyside liner in the pair of side seams in the back waist region.

In another aspect, the article defines a front waist region, a back waist region, a crotch region that extends between and connects the front and back waist regions, a longitudinal direction and a lateral direction. The article includes:

A stretchable absorbent assembly defining a pair of longitudinally opposed end margins and a pair of laterally opposed side margins. The stretchable absorbent assembly includes: i) a stretchable outercover; ii) a stretchable bodyside liner joined to the stretchable outercover in a superposed relation to form a pair of absorbent assembly side seams and a pair of absorbent assembly end seams; iii) a first elastomeric suspension member attached to the absorbent assembly in the front waist region; iv) a second elastomeric suspension member attached to the absorbent assembly in the back waist region; and v) an absorbent body disposed between the outercover and the bodyside liner of the absorbent assembly and joined to the first elastomeric suspension member and the second elastomeric suspension member. The absorbent body is configured to float between the outercover and the bodyside liner.

In yet another aspect, the article defines a front waist region, a back waist region, a crotch region that extends between and connects the front and back waist regions, a longitudinal direction and a lateral direction, and a pair of longitudinally opposed end margins and a pair of laterally opposed side margins. The article includes A stretchable outercover and a stretchable bodyside liner joined to the stretchable outercover in a superposed relation. The outercover and the bodyside liner are joined along at least a portion of the end margins to provide a front waist seam and a back waist seam. Further, the outercover and the bodyside liner are joined along at least a portion of each of the side margins in each of the front waist region and the back waist region to provide a pair of side seams in the front waist region and the back waist region. The article also includes an absorbent body disposed between the outercover and the bodyside liner, the absorbent body defining a pair of longitudinally opposed absorbent end edges and a pair of laterally opposed absorbent side edges. In addition, the article includes at least one elastomeric suspension member attached to each of the absorbent side edges of the absorbent body in the front waist region. The at least one elastomeric suspension member is sandwiched between the outercover and the bodyside liner in the front waist seam. Finally, the article also includes at least one elastomeric suspension member attached to each of the absorbent side edges of the absorbent body in the rear waist region. The at least one elastomeric suspension member is sandwiched between the outercover and the bodyside liner in the back waist seam.

Other features of the invention will be in part apparent and in part pointed out hereinafter.

### BRIEF DESCRIPTION OF THE DRAWINGS

FIG. 1 representatively illustrates a side perspective of an article of the present invention represented in the form of a pair of training pants having a mechanical fastening system fastened on one side of the training pants and unfastened on the opposite side thereof;

FIG. 2 representatively illustrates a bottom plan view of the training pants of FIG. 1 with the pants in an unfastened, unfolded and laid flat condition, and showing the surface of the training pants that faces away from the wearer;

FIG. 3 representatively illustrates a top plan view similar to FIG. 2 showing the surface of the training pants that faces the wearer when worn and with portions cut away to show underlying features;

FIG. 4 representatively illustrates a top plan view of another aspect of the present invention showing the surface of the training pants that faces the wearer and with portions cut away to show underlying features;

FIG. 5 representatively illustrates a side perspective of another aspect of the present invention shown in the form of a pair of training pants having a pair of separately attached side panels and a mechanical fastening system fastened on one side of the training pants and unfastened on the opposite side thereof;

FIG. 6 representatively illustrates a bottom plan view of the training pants of FIG. 5 with the pants in an unfastened, unfolded and laid flat condition, and showing the surface of the training pants that faces away from the wearer; and

FIG. 7 representatively illustrates a top plan view similar to FIG. 2 showing the surface of the training pants that faces the wearer when worn and with portions cut away to show underlying features.

Corresponding reference characters indicate corresponding parts throughout the several views of the drawings.

### DETAILED DESCRIPTION OF THE INVENTION

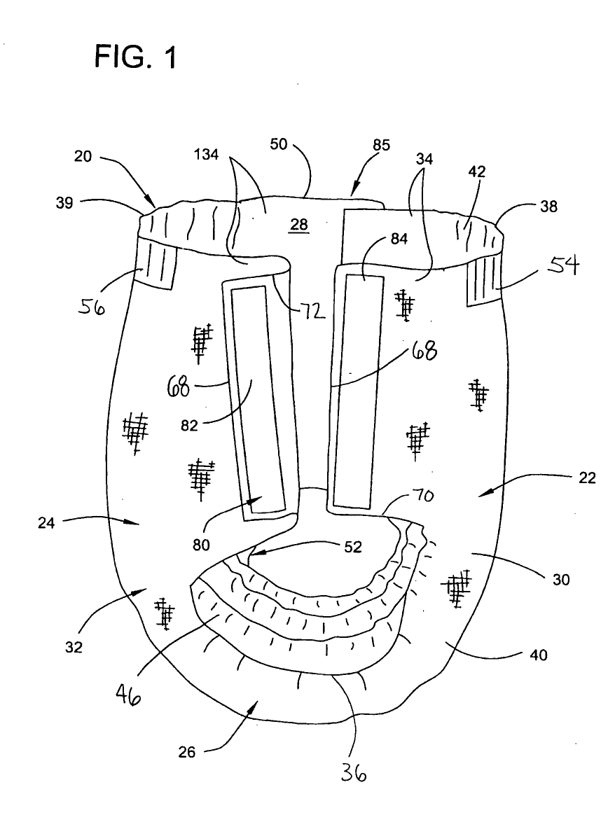

Referring now to the drawings and in particular to FIG. 1, an absorbent article of the present invention is representatively illustrated therein in the form of children's toilet training pants and is indicated in its entirety by the reference numeral 20. The absorbent article 20 may or may not be disposable, which refers to articles that are intended to be discarded after a limited period of use instead of being laundered or otherwise conditioned for reuse. It is understood that the present invention is suitable for use with various other absorbent articles intended for personal wear, including but not limited to diapers, feminine hygiene products, incontinence products, medical garments, surgical pads and bandages, other personal care or health care garments, and the like without departing from the scope of the present invention.

By way of illustration only, various materials and methods for constructing training pants such as the pants 20 of the various aspects of the present invention are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference to the extent they are consistent (i.e., not in conflict) herewith.

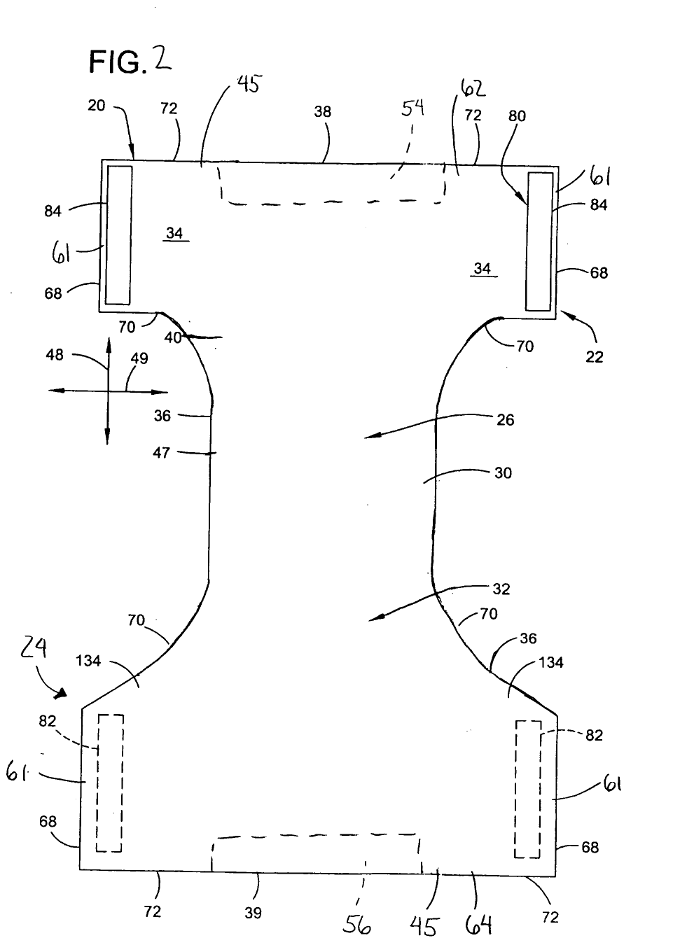

A pair of training pants 20 is representatively illustrated in FIG. 1 in a partially fastened condition. The pants 20 define a longitudinal direction 48 and a lateral direction 49 perpendicular to the lateral direction (FIGS. 2-4, 6 and 7). The pants 20 further define a pair of longitudinal end regions, otherwise referred to herein as a front waist region 22 and a back waist region 24, and a center region, otherwise referred to herein as a crotch region 26, extending longitudinally between and interconnecting the front and back waist regions 22, 24. The pant 20 also defines an inner surface 28 adapted in use (e.g., positioned relative to the other components of the pants 20) to be disposed toward the wearer, and an outer surface 30 opposite the inner surface. The front and back waist regions 22, 24 are those portions of the pants 20, which when worn, wholly or partially cover or encircle the waist or mid-lower torso of the wearer. The crotch region 26 generally is that portion of the pants 20 which, when worn, is positioned between the legs of the wearer and covers the lower torso and crotch of the wearer. The training pants 20 have a pair of laterally opposite side edges 36 and a pair of longitudinally opposite waist edges, respectively designated front waist edge 38 and back waist edge 39.

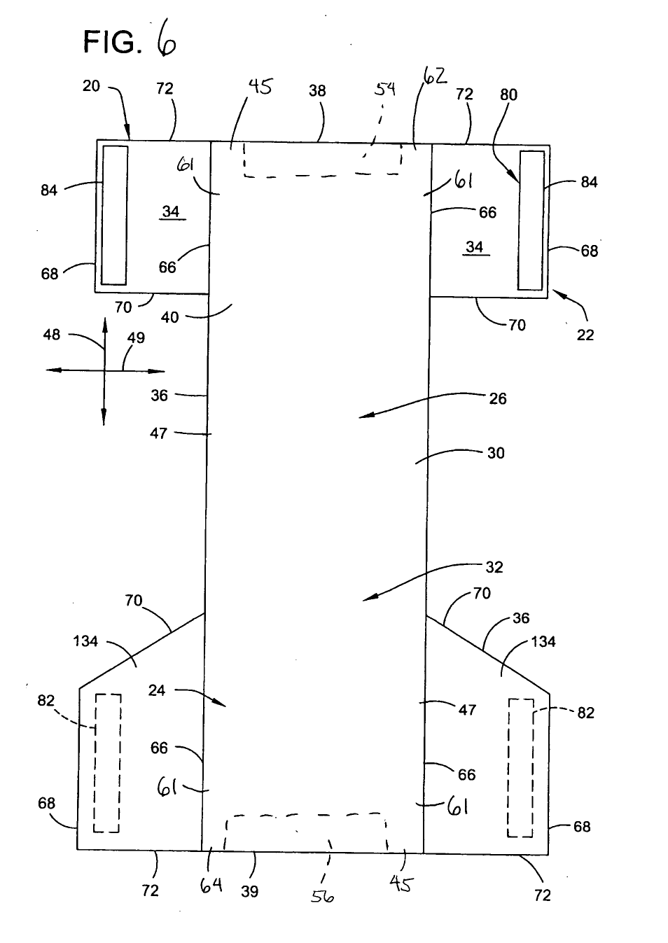

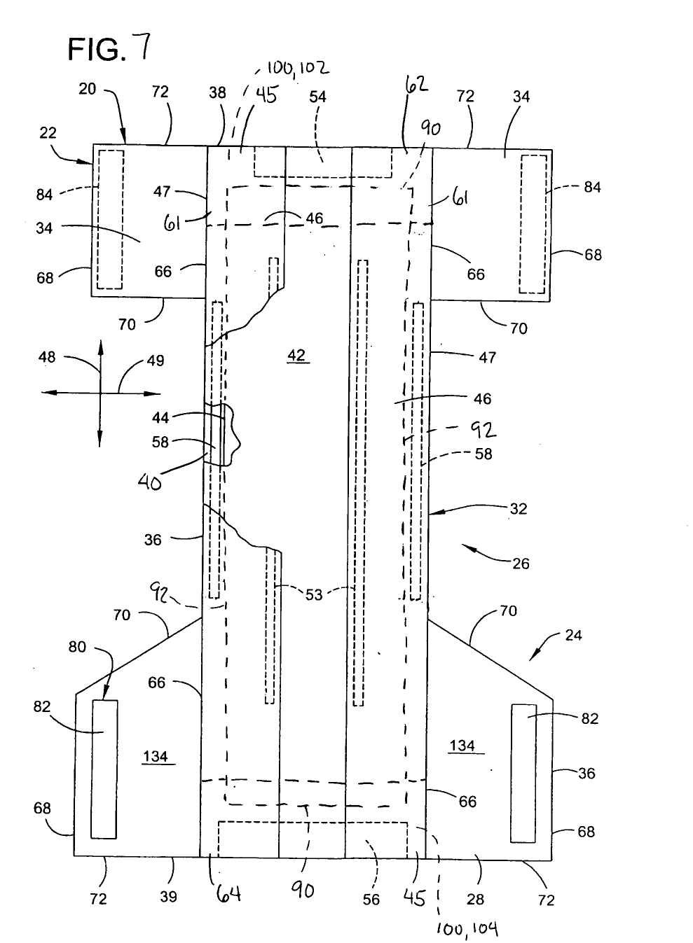

The illustrated pants 20 may include a central absorbent assembly, generally indicated at 32, a pair of laterally opposite front side panels 34 extending laterally outward at the front waist region 22 and a pair of laterally opposite back side panels 134 extending laterally outward at the back waist region 24. The central absorbent assembly 32 is illustrated in FIGS. 6 and 7 as being generally rectangular. However, it is contemplated that the absorbent assembly 32 may be other than rectangular, such as hourglass shaped as representatively illustrated in FIGS. 2-4, T-shaped, I-shaped, and the like without departing from the scope of this invention.

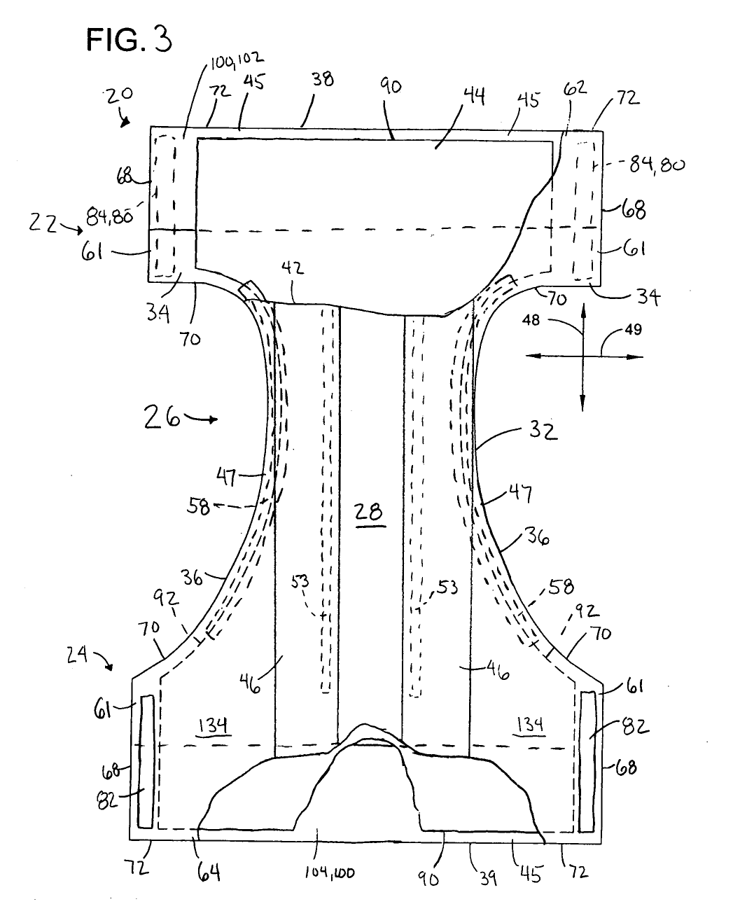

The central absorbent assembly 32 of the illustrated aspect has a pair of longitudinally opposed end margins 45, which form at least portions of the front and back waist edges 38, 39, and a pair of laterally opposed side margins 47 which form at least portions of the pant side edges 36 (FIGS. 2 and 3). For further reference, arrows 48 and 49 depict the orientation of the longitudinal direction and the transverse or lateral direction, respectively, of the training pants 20.

Still referring to FIGS. 1-3, the central absorbent assembly 32 includes an outercover 40 and a bodyside liner 42 (FIGS. 1 and 3) that may be joined to the outercover 40 in a superposed relation therewith by adhesives, ultrasonic bonds, thermal bonds or other conventional techniques. The liner 42 may suitably be joined to the outercover 40 along at least a portion of the end margins 45 in the front waist region 22 and the back waist region 24 to provide a front waist seam 62 and a back waist seam 64. In addition, the liner 42 may suitably be joined to the outercover 40 along at least a portion of the side margins 47 in the front waist region 22 and the back waist region 24 to provide a pair of side seams 61 in the front waist region 22 and the back waist region 24. The liner 42 can be generally adapted, i.e., positioned relative to the other components of the pants 20, to be disposed toward the wearer's skin during wear of the pants. The absorbent assembly 32 may further include an absorbent body 44 (FIGS. 3, 4, and 7) disposed between the outercover 40 and the bodyside liner 42 for absorbing liquid body exudates exuded by the wearer, and may further include a pair of containment flaps 46 (FIGS. 1, 3, 5, and 7) secured to the bodyside liner 42 for inhibiting the lateral flow of body exudates.

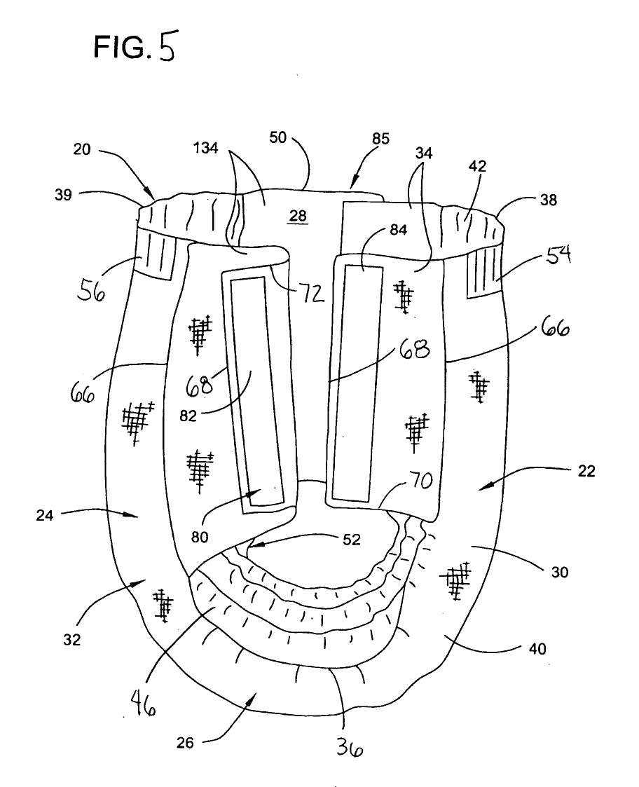

With the training pants 20 in the fastened position as partially illustrated in FIGS. 1 and 5, the front and back side panels 34, 134 can be connected together by a fastening system 80 to define a three-dimensional pants configuration having a waist opening 50 and a pair of leg openings 52. The front and back side panels 34 and 134, upon wearing of the pants 20, thus include the portions of the training pants 20 which are positioned on the hips of the wearer. The waist edges 38 and 39 of the training pants 20 are configured to encircle the-waist of the wearer and together define a waist opening 50 of the pants.

As representatively illustrated in FIGS. 3 and 7, the training pants 20 of the present invention may include containment flaps 46. A flap elastic member 53 can be operatively joined with each containment flap 46 in any suitable manner as is well known in the art. The elasticized containment flaps 46 define a partially unattached edge which assumes an upright configuration in at least the crotch region 26 of the training pants 20 to form a seal against the wearer's body. The containment flaps 46 can be located along the side margins 47, and can extend longitudinally along the entire length of the absorbent assembly 32 or may extend only partially along the length of the absorbent assembly. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the training pants 20 may also suitably include a front waist elastic member 54 (FIGS. 1, 2, and 5-7), a rear waist elastic member 56, and leg elastic members 58 (FIGS. 3, 4, and 7), as are known to those skilled in the art. The waist elastic members 54 and 56 can be operatively joined to the outercover 40 and/or the bodyside liner 42 along the opposite end margins 45, and can extend over part or all of the waist edges 38, 39. The leg elastic members 58 can be operatively joined to the outercover 40 and/or the bodyside liner 42 along the opposite side margins 47 and positioned in the crotch region 26 of the training pants 20.

The flap elastic members 53, the waist elastic members 54 and 56, and the leg elastic members 58 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic retractive forces are imparted to the substrate. In one particular aspect, for example, the leg elastic members 58 may include a plurality of dry-spun coalesced multifilament spandex elastomeric threads sold under the trade name LYCRA® and available from E. I. Du Pont de Nemours and Company, Wilmington, Del., U.S.A.

In configurations where the side panels 34, 134, are separately attached (FIGS. 5-7) the panels can be permanently bonded along seams 66 to the central absorbent assembly 32 in the respective front and back waist regions 22 and 24. More particularly, as seen best in FIGS. 6 and 7, the front side panels 34 can be permanently bonded to and extend transversely outward beyond the side margins 47 of the absorbent assembly 32 at the front waist region 22, and the back side panels 134 can be permanently bonded to and extend transversely outward beyond the side margins of the absorbent assembly at the back waist region 24. The side panels 34 and 134 may be bonded to the absorbent assembly 32 using attachment means known to those skilled in the art such as adhesive, thermal or ultrasonic bonding.

Alternatively, the side panels 34 and 134 can be formed as an integral portion of a component of the absorbent assembly 32 as representatively illustrated in FIGS. 1-4. For example, the side panels 34, 134 can include a generally wider portion of the outercover 40, the bodyside liner 42, and/or other components of the absorbent assembly 32. In any event, the front and back side panels 34 and 134 can be permanently bonded together to form the three-dimensional configuration of the pants 20, or be releasably connected with one another such as by the fastening system 80 of the illustrated aspects.

The front and back side panels 34, 134 each have a longitudinal outer-edge 68, a leg end edge 70 disposed toward the longitudinal center of the training pants 20, and a waist end edge 72 disposed toward a longitudinal end of the training pants. The leg end edges 70 and outer edges 68 of the side panels 34 and 134 form part of the pant side edges 36 of the training pants 20. In configurations where the side panels 34 and 134 are integral, the side margins 47 provide the outer edge 68, leg end edges 70 and the pant side edges 36. The leg end edges 70 of the illustrated aspects may be suitably curved and/or angled relative to the lateral direction 49 to provide a better fit around the wearer's legs. However, it is understood that only one of the leg end edges 70 may be curved or angled, such as the leg end edge of the back waist region 24, or alternatively, neither of the leg end edges may be curved or angled, without departing from the scope of this invention. The waist end edges 72 are suitably parallel to the transverse axis 49. The waist end edges 72 of the front side panels 34 form part of the front waist edge 38 of the training pants 20, and the waist end edges 72 of the back side panels 134 form part of the back waist edge 39 of the pants. In configurations where the side panels 34 and 134 are integral, the end margins 45 provide the waist end edges 72 and the pant waist edges 38 and 39.

In configurations where the side panels 34, 134 are separately attached, the side panels may be provided by an elastic material capable of stretching at least in a direction generally parallel to the lateral direction 49 of the training pants 20. Suitable elastic materials, as well as one process of incorporating elastic side panels into training pants, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. In particular aspects, the elastic material may include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference to the extent that they are consistent (i.e., not in conflict) herewith. Alternatively, the side panel material may include other woven or non-woven materials, such as those described later herein as being suitable for construction of the outercover 40 and/or the bodyside liner 42; mechanically pre-strained composites; or stretchable but inelastic materials.

The fastening system 80 may include laterally opposite first fastening components 82 adapted for refastenable engagement to corresponding second fastening components 84. In one aspect, a front or outer surface of each of the fastening components 82, 84 includes a plurality of engaging elements. The engaging elements of the first fastening components 82 are adapted to repeatedly engage and disengage corresponding engaging elements of the second fastening components 84 to releasably secure the pants 20 in its three-dimensional configuration.

The fastening components 82, 84 may be any refastenable fasteners suitable for absorbent articles, such as adhesive fasteners, cohesive fasteners, mechanical fasteners, or the like. In particular aspects the fastening components include mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

In the illustrated aspect, the first fastening components 82 include loop fasteners and the second fastening components 84 include complementary hook fasteners. Alternatively, the first fastening components 82 may include hook fasteners and the second fastening components 84 may be complementary loop fasteners. In another aspect, the fastening components 82, 84 can be interlocking similar surface fasteners, or adhesive and cohesive fastening elements such as an adhesive fastener and an adhesive-receptive landing zone or material; or the like. Although the training pants 20 illustrated in FIG. 1 indicate the back side panels 134 overlapping the front side panels 34 upon connection thereto, which is convenient, the training pants 20 can also be configured so that the front side panels 34 overlap the back side panels 134 when connected. One skilled in the art will recognize that the shape, density and polymer composition of the hooks and loops may be selected to obtain the desired level of engagement between the fastening components 82, 84. Optionally, either one or both of the fastening components 82, 84 may be provided by one of the inner or outer surfaces 28 and 30 of the side panels 34 and 134. When engaged, the fastening components 82, 84 of the illustrated aspect define refastenable engagement seams 85 (FIGS. 1 and 5). Suitable fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

The outercover 40 suitably includes a material which is substantially liquid impermeable. The outercover 40 can be a single layer of liquid impermeable material, or alternatively can be a multi-layered laminate structure in which at least one of the layers is liquid impermeable. For instance, the outercover 40 can include a liquid permeable outer layer and a liquid impermeable inner layer that are suitably joined together by a laminate adhesive, ultrasonic bonds, thermal bonds, or the like. Suitable laminate adhesives, which can be applied continuously or intermittently as beads, a spray, parallel swirls, or the like, can be obtained from Bostik Findley Adhesives, Inc., of Wauwatosa, Wisc., U.S.A., or from National Starch and Chemical Company, Bridgewater, N.J. U.S.A. The liquid permeable outer layer can be any suitable material and is desirably one that provides a generally cloth-like texture. One example of such a material is a 20 gsm (grams per square meter) spunbond polypropylene nonwoven web. The outer layer may also be made of those materials of which the liquid permeable bodyside liner 42 is made.

The inner layer of the outercover 40 can be both liquid and vapor impermeable, or it may be liquid impermeable and vapor permeable. The inner layer can be manufactured from a thin plastic film, although other flexible liquid impermeable materials may also be used. The inner layer, or the liquid impermeable outercover 40 when a single layer, prevents waste material from wetting articles, such as bed sheets and clothing, as well as the wearer and caregiver. A suitable liquid impermeable film for use as a liquid impermeable inner layer, or a single layer liquid impermeable outercover 40, is a 0.02 millimeter polyethylene film commercially available from Pliant Corporation of Schaumburg, Ill., U.S.A.

In particular aspects, the outercover 40 may be stretchable, and even more particularly the outercover may be elastomeric. As used herein, the term “stretchable” refers to a material that may be extensible and/or elastomeric. That is, the material may be extended, deformed or the like, without breaking, and may or may not significantly retract after removal of an extending force. The terms “elastomeric” or “elastic” are used interchangeably herein and refer to that property of a material where upon removal of an elongating force, the material is capable of recovering to substantially its unstretched size and shape or the material exhibits a significant retractive force. The term “extensible” refers to that property of a material where upon removal of an elongating force, the material experiences a substantially permanent deformation or the material does not exhibit a significant retractive force. In particular, elastomeric materials utilized in connection with the present invention may be elongated/extended or stretched in at least one direction without breaking by at least 25% (to at least 125% of its initial unstretched length) in at least one direction, suitably by at least 50% (to at least 150% of its initial unstretched length) and which will recover, upon release of the applied stretching or biasing force, at least 10% of their elongation. It is generally advantageous that the elastomeric material or composite be capable of being elongated by at least 100%, more desirably by at least 200%, of its relaxed length and recover at least 30% and more desirably 50% of its elongation upon release of a stretching, biasing force, within about one minute.

Similarly, extensible or elongatable materials of the present invention may be capable of elongating/extending or stretching in at least one direction without breaking by at least 25% (to at least 125% of its initial unstretched length) in at least one direction, suitably by at least 50% (to at least 150% of its initial unstretched length), more suitably by at least 100% (to at least 200% of its initial unstretched length). As an example, an extensible material having an initial unstretched length of 3 inches (7.6 centimeters) may be stretched without breaking to a stretched length of at least 3.75 inches (9.5 centimeters) in at least one direction (for the “by at least 25%” value).

The outercover 40 may be constructed of a single layer, multiple layers, laminates, spunbond fabrics, films, meltblown fabrics, elastic netting, microporous web, bonded carded webs or foams provided by elastomeric or polymeric materials. Elastomeric non-woven laminate webs can include a non-woven material joined to one or more gatherable non-woven webs, films, or foams. Stretch Bonded Laminates (SBL) and Neck Bonded Laminates (NBL) are examples of elastomeric composites. Non-woven fabrics are any web of material which has been formed without the use of textile weaving processes which produce a structure of individual fibers that are interwoven in an identifiable repeating manner.

Examples of suitable materials are spunbond-meltblown fabrics, spunbond-meltblown-spunbond fabrics, spunbond fabrics, or laminates of such fabrics with films, foams, or other nonwoven webs. Elastomeric materials may include cast or blown films, foams, or meltblown fabrics composed of polyethylene, polypropylene, or polyolefin copolymers, as well as combinations thereof. The elastomeric materials may include PEBAX elastomer (available from AtoChem located in Philadelphia, Pa.), HYTREL elastomeric polyester (available from E. I. DuPont de Nemours of Wilmington, Del.), KRATON elastomer (available from Kraton Polymers of Houston, Tex.), or strands of LYCRA elastomer (available from E. I. DuPont de Nemours of Wilmington, Del.), or the like, as well as combinations thereof. The outercover 40 may include materials that have elastomeric properties through a mechanical process, printing process, heating process, or chemical treatment. For examples such materials may be apertured, creped, neck-stretched, heat activated, embossed, and micro-strained; and may be in the form of films, webs, and laminates.

In particular aspects of the invention, the outercover 40 may include a 0.4 ounces per square yard (osy) (13.6 grams per square meter (gsm)) basis weight layer of G2760 KRATON elastomer strands adhesively laminated with a 0.3 gsm layer of adhesive between two facings. Each facing can be composed of a thermal point bonded bicomponent spunbond non-woven fibrous web having a 0.7 osy (23.7 gsm) basis weight. The adhesive is similar to an adhesive which is supplied by AtoFindley Adhesive and designated as H2525 A, and the elastomer strands are placed and distributed to provide approximately 12 strands of KRATON elastomer per inch (2.54 cm) of lateral width of the outercover 40.

Alternatively, the outercover 40 may include a woven or non-woven fibrous web layer which has been totally or partially constructed or treated to impart the desired levels of liquid impermeability to selected regions that are adjacent or proximate the absorbent body. For example, the outercover 40 may include a gas-permeable, non-woven fabric layer laminated to a polymer film layer which may or may not be gas-permeable. Other examples of fibrous, cloth-like outercover 40 materials can include a stretch thinned or stretch thermal laminate material composed of a 0.6 mil (0.015 mm) thick polypropylene blown film and a 0.7 osy (23.8 gsm) polypropylene spunbond material (2 denier fibers).

Suitable materials for a biaxially stretchable outercover 40 include biaxially stretchable material and biaxially elastic stretchable material. One example of a suitable outercover material can include a 0.3 osy polypropylene spunbond that is necked 60% in the lateral direction 40 and creped 60% in the longitudinal direction 48, laminated with 3 grams per square meter (gsm) Findley 2525A styrene-isoprene-styrene based adhesive to 8 gsm PEBAX 2533 film with 20% TiO₂ concentrate. The outercover 40 can suitably be stretched, laterally and/or longitudinally, by at least 30% (to at least 130% of an initial (unstretched) width and/or length of the outercover 40). More suitably, the outercover 40 can be stretched laterally and/or longitudinally, by at least 50% (to at least 150% of the unstretched width or length of the outercover 40). Even more suitably, the outercover 40 can be stretched, laterally and/or longitudinally, by at least 100% (to at least 200% of the unstretched width or length of the outercover 40). Tension force in the outercover 42 at 50% extension is suitably between 50 and 1000 grams, more suitably between 100 and 600 grams, as measured on a 3 inch (7.62 cm) wide piece of the outercover material.

Another example of a suitable material for a biaxially stretchable outercover 40 is a breathable elastic film/nonwoven laminate, described in U.S. Pat. No. 5,883,028, issued to Morman et al., incorporated herein by reference to the extent that it is consistent (i.e. not in conflict) herewith. Examples of materials having two-way stretchability and retractability are disclosed in U.S. Pat. No. 5,116,662 issued to Morman and U.S. Pat. No. 5,114,781 issued to Morman, both of which are hereby incorporated herein by reference to the extent that it is consistent (i.e., not in conflict) herewith. These two patents describe composite elastic materials capable of stretching in at least two directions. The materials have at least one elastic sheet and at least one necked material, or reversibly necked material, joined to the elastic sheet at least at three locations arranged in a nonlinear configuration, so that the necked, or reversibly necked, web is gathered between at least two of those locations.

The outercover 40 can be suitably sized (e.g., in length and width) larger than the absorbent body 44 to extend outward beyond the periphery thereof. For example, the outercover 40 may extend outward beyond the absorbent body periphery a distance in the range of about 1.3 centimeters to about 2.5 centimeters (about 0.5 to 1 inch). Alternatively, the outercover 40 may extend a greater amount or a lesser amount beyond the periphery of the absorbent body 44 as is known in the art.

The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent body 44. A suitable bodyside liner 42 may be manufactured from a wide selection of web materials, such as porous foams, reticulated foams, apertured plastic films, woven and non-woven webs, or a combination of any such materials. For example, the bodyside liner 42 may include a meltblown web, a spunbonded web, or a bonded-carded-web composed of natural fibers, synthetic fibers or combinations thereof. The bodyside liner 42 may be composed of a substantially hydrophobic material, and the hydrophobic material may optionally be treated with a surfactant or otherwise processed to impart a desired level of wettability and hydrophilicity.

The bodyside liner 42 may also be stretchable, and more suitably it may be, elastomeric. Suitable elastomeric materials for construction of the bodyside liner 42 can include elastic strands, LYCRA elastics, cast or blown elastic films, nonwoven elastic webs, meltblown or spunbond elastomeric fibrous webs, as well as combinations thereof. Examples of suitable elastomeric materials include KRATON elastomers, HYTREL elastomers, ESTANE elastomeric polyurethanes (available from B.F. Goodrich and Company of Cleveland, Ohio), or PEBAX elastomers.

As an additional example, in one aspect the bodyside liner 42 suitably includes a non-woven, spunbond polypropylene fabric composed of about 2 to 3 denier fibers formed into a web having a basis weight of about 12 gsm which is necked approximately 60 percent. Strands of about 9 gsm KRATON G2760 elastomer material placed eight strands per inch (2.54 cm) are adhered to the necked spunbond material. The fabric is surface treated with an operative amount of surfactant, such as about 0.6 percent AHCOVEL Base N62 surfactant, available from ICI Americas, a business having offices in Wilmington, Del., U.S.A. The surfactant can be applied by any conventional means, such as spraying, printing, brush coating or the like. Other suitable materials may be extensible biaxially stretchable materials, such as a neck stretched/creped spunbond. The bodyside liner 42 can also be made from extensible materials as are described in U.S. patent application Ser. No. 09/563,417 filed on May 3, 2000 by Roessler et al. The bodyside liner 42 can also be made from biaxially stretchable materials as are described in U.S. patent application Ser. No. 09/698,512 filed on Oct. 27, 2000 by Vukos et al.

The liner 42 can suitably be stretched, laterally and/or longitudinally, by at least 30% (to at least 130% of an initial (unstretched) width and/or length of the liner 42). More suitably, the liner 42 can be stretched laterally and/or longitudinally, by at least 50% (to at least 150% of the unstretched width or length of the liner 42). Even more suitably, the liner 42 can be stretched, laterally and/or longitudinally, by at least 100% (to at least 200% of the unstretched width or length of the liner 42). Tension force in the liner 42 at 50% extension is suitably between 50 and 1000 grams, more suitably between 100 and 600 grams, as measured on a 3 inch (7.62 cm) wide piece of the liner material.

The absorbent body 44 may be disposed between the outercover 40 and the bodyside liner 42 and may define a pair of longitudinally opposed absorbent end edges 90 and a pair of laterally opposed absorbent side edges 92 (FIGS. 3, 4, 7). The absorbent body 44 can be any structure or combination of components which are generally compressible, conformable, non-irritating to a wearer's skin, and capable of absorbing and retaining liquids and certain body wastes. For example, the absorbent body 44 may include an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular aspect, the absorbent web material is a matrix of cellulosic fluff and superabsorbent hydrogel-forming particles. The cellulosic fluff may include a blend of wood pulp fluff. One preferred type of fluff is identified with the trade designation CR 1654, available from U.S. Alliance of Childersburg, Ala., USA, and is a bleached, highly absorbent sulfate wood pulp containing primarily soft wood fibers. The absorbent materials may be formed into a web structure by employing various conventional methods and techniques. For example, the absorbent web may be formed with a dry-forming technique, an air forming technique, a wet-forming technique, a foam-forming technique, or the like, as well as combinations thereof. Methods and apparatus for carrying out such techniques are well known in the art. Furthermore, the absorbent body structure may itself encompass multiple layers in the Z direction. Such multiple layers may take advantage of differences in absorbency capacity, such as by placing a lower capacity absorbent material layer closer to the liner 42 and a higher capacity absorbent material closer to the outercover layer 40. Likewise, discrete portions of an absorbent body single-layered structure may encompass higher capacity absorbents, and other discrete portions of the structure may encompass lower capacity absorbents.

As a general rule, the superabsorbent material is present in the absorbent web in an amount of from about 0 to about 90 weight percent based on total weight of the web. The web may have a density within the range of about 0.10 to about 0.35 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a suberabsorbent material is capable of absorbing at least about 10 times its weight in liquid, and desirably is capable of absorbing more than about 25 times its weight in liquid. Suitable superabsorbent materials are readily available from various suppliers. For example, Favor 880, and Favor 9543 superabsorbents are available from Stockhausen GmbH of Germany.

After being formed or cut into a desired shape, the absorbent web material may be wrapped or encompassed by a suitable tissue wrap that aids in maintaining the integrity and shape of the absorbent body 44.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in certain aspects, thermoplastic polymers can be utilized. Some examples of suitable thermoplastics that can be utilized include polyolefins, such as polyethylene, polypropylene, polybutylene and the like; polyamides; and polyesters. In one aspect, the thermoplastic polymer is polypropylene. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein by reference to the extent they are consistent (i.e., not in conflict) herewith.

In a particular aspect of the absorbent article of the present invention, the absorbent body 44 may also be elastomeric. For example, in the aspect as illustrated in FIG. 1 the entire central absorbent assembly 32 may be stretchable. In such a configuration, the absorbent body 44 may be stretchable so as not to inhibit the stretchability of other components.

For this purpose, the absorbent web material can include elastomeric fibers in an amount which is at least a minimum of about 2 wt %. The amount of elastomeric fibers can alternatively be at least about 3 wt %, and can optionally be at least about 5 wt % to provide improved performance. In addition, the amount of elastomeric fibers can be not more than about 60 wt %. Alternatively, the amount of elastomeric fibers can be not more than about 45 wt %, and optionally, can be not more than about 30 wt % to provide improved benefits. These values may impact the absorbent body 44 by affecting the desired levels of stretchability and structural stability without excessively degrading the physical properties or the liquid-management properties of the absorbent body. An absorbent web material with an excessively low proportion of elastomeric fibers may be insufficiently stretchable, and a web material with an excessively high proportion of elastomeric fibers may exhibit an excessive degradation of its absorbency functionalities, such as poor intake, poor distribution, poor retention of liquid.

The absorbent body 44 may include an elastomeric coform absorbent web material. Such materials are described for instance in U.S. Pat. Nos. 6,231,557 B1 and 6,362,389 B1, which are each incorporated by reference herein to the extent they are consistent (i.e., not in conflict) herewith. In particular aspects, the elastomeric coform material can have an overall coform basis weight which is at least a minimum of about 50 g/m². The coform basis weight can alternatively be at least about 100 g/m² and can optionally be at least about 200 g/m² to provide improved performance. In addition, the coform basis weight can be not more than about 1200 g/m². Alternatively, the coform basis weight can be not more than about 900 g/m², and optionally, can be not more than about 800 g/m² to provide improved benefits. These values can provide the absorbent body structure with the desired stretchability and structural stability without excessively degrading the physical properties or the liquid-management functionalities of the absorbent body structure. For example, retention portions having excessively low proportions of elastomeric coform material may not be sufficiently stretchable. Conversely, an absorbent web material having excessively large amounts of elastomeric coform materials can exhibit an excessive degradation of their absorbency functionalities, such as an excessive degradation of intake, distribution and/or retention properties.

Other examples of usable elastomeric absorbent bodies are described in international patent application WO 03/051254 and U.S. Pat. Nos. 5,964,743, 5,645,542, 6,231,557, and 6,362,389 B1, each of which are incorporated by reference herein to the extent they are consistent (i.e., not in conflict) herewith.

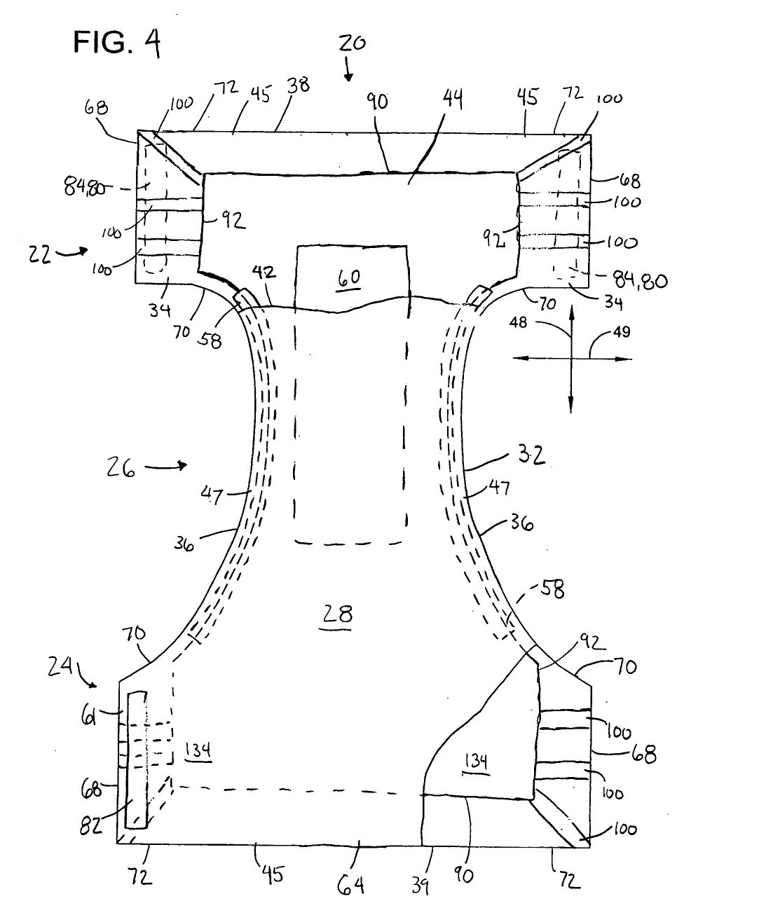

In certain aspects, a surge management layer 60 may be optionally located adjacent the absorbent body 44 and attached to various components in the article 20 such as the absorbent body 44 or the bodyside liner 42 by methods known in the art, such as by adhesive (FIG. 4). A surge management layer 60 helps to decelerate and diffuse surges or gushes of liquid that may be rapidly introduced into the absorbent body of the article. Desirably, the surge management layer can rapidly accept and temporarily hold the liquid prior to releasing the liquid into the storage or retention portions of the absorbent body. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference herein to the extent they are consistent (i.e., not in conflict) herewith.

The training pants 20 of the various aspects of the present invention may further include at least one suspension member 100 attached to the absorbent body 44 in one of the waist regions 22, 24. As used herein, “attach,” and its variations, such as “attached,” and “attachment,” and “join” and its variations, such as “joined” or “joinder” may indicate a direct connection of multiple elements, an indirect connection of multiple elements (such as via at least one intermediate element), or an integral association of elements.

As will be discussed in greater detail below, the suspension members 100 may be suitably stretchable, and still more suitably, elastomeric. For example, as representatively illustrated in FIGS. 3 and 7, the training pants 20 may include a first suspension member 102 attached to the absorbent body in the front waist region 24 and a second suspension member 104 attached to the absorbent body 44 in the back waist region 24. The suspension members 100 may be sandwiched between the outercover 40 and the liner 42 in at least one of the seams 61, 62, 64 to join the absorbent body 44 to the central absorbent assembly 32.

Accordingly, the absorbent body 44 is secured within the central absorbent assembly, while yet is configured to “float” between the outercover 40 and the bodyside liner 42. That is, the absorbent body 44 is suspended relative to the outercover 40 and liner 42 and is therefore slidably contained between the outercover 40 and the liner 42 while remaining attached to the central absorbent assembly 32 via the suspension members 100. This is particularly useful in configurations where the outercover 40 and the liner 42 are stretchable, as in such arrangements the extent to which the stretch of the outercover 40 and the liner may be limited by the absorbent body 44 is minimized. Further, the integrity of the absorbent body 44 will be better maintained during donning of the article as well as during wear as the suspension members 100 aid in maintaining the positioning of the body 44 within the absorbent assembly 32. Moreover, the suspension members 100 are capable of isolating the absorbent body 44 from many of the forces that are exerted on the absorbent assembly 32, particularly in configurations where the suspension members 100 are stretchable. Further, in certain instances where the absorbent body 44 may be subjected to sagging or drooping in the crotch region 26 (for example, from repeated insults), the suspension members 100 may isolate the absorbent assembly 32 from these forces, thus resulting in improved fit and performance of the pants 20.

The suspension members 100 may be attached to the absorbent body 44 by any suitable means known in the art, including adhesives, molten thermoplastic material that is allowed to fuse upon solidification, ultrasonic bonding, or pressure bonding and the like or combinations thereof. As representatively illustrated in FIGS. 3 and 4, the absorbent body 44 may be placed upon the suspension member for attachment, and thus be disposed toward the inner surface 28 relative to the suspension member 100. Alternatively, a portion of the suspension member 100 may be inserted into the absorbent body 44 or laid upon the absorbent body 44 (that is, the suspension member 100 may be disposed toward the inner surface 28 relative to the absorbent body 44) for attachment, or a combination of such configurations may be employed. Desirably, the absorbent body 44 is disposed toward the inner surface 28 relative to the suspension member 100 for improved performance and manufacturability. Specifically, in such a configuration, the suspension member 100 is less likely to hamper the intake of fluid by the absorbent body 44, or reduce the dispersal of fluid throughout the absorbent body 44.

As a portion of the suspension members 100 is attached to the absorbent body 44, an opposing portion of the suspension members 100 may be attached to the absorbent assembly 32 by being sandwiched between the outercover 40 and the liner 42. In particular, as representatively illustrated in FIGS. 3 and 4, the suspension members 100 may be sandwiched between the outercover 40 and the liner 42 in the front waist seam 62, the back waist seam 64, or the side seams 61, or combinations thereof. Accordingly, the suspension members 100 may be held in place in the seams 61, 62 and 64 by way of the joinder of the outercover 40 and the liner 42, or additional attachment may be employed to hold the suspension members 100 in the seams 61, 62 and 64 such as adhesives, molten thermoplastic material that is allowed to fuse upon solidification, ultrasonic bonding, or pressure bonding, stitching, and the like or combinations thereof.

In a particular aspect, the pants 20 may include a first suspension member 102 located in the front waist region 22 and a second suspension member 104 located in the back waist region 24. As representatively illustrated in FIGS. 3 and 7, the training pants 20 of the present invention may include at least a first suspension member 102 attached to the absorbent body 44 in the front waist region 22, and a second suspension member 104 attached to the absorbent body 44 in the back waist region 24. The suspension members 102 and 104 may extend from the absorbent body 44 and be attached to the absorbent assembly 32 in at least the side seams 61. For example, the first suspension member 102 may extend from the absorbent body 44 in the front waist region 22 and may be attached to the absorbent assembly 32 in the side seams 61 in the front waist region 22. Specifically, the first suspension member 102 may be sandwiched between the outercover 40 and the bodyside liner 42 in the side seams 61 in the front waist region 22. Similarly, the second suspension member 104 may extend from the absorbent body 44 in the back waist region 24 and may be attached to the absorbent assembly 32 in the side seams 61 in the back waist region 24.

In particular, the second suspension member 104 may be sandwiched between the outercover 40 and the bodyside liner 42 in the side seams 61 in the back waist region 24. As such, the absorbent body 44 may be configured to float between the outercover 40 and the bodyside liner 42. Further, in such an arrangement, the suspension members 102 and 104 can assist in maintaining the positioning of the absorbent body 44 while the absorbent assembly 32 is subjected to forces in the lateral direction 49. Further, as illustrated in FIGS. 4 and 7, the suspension members 102 and 104 may extend in the longitudinal direction 48 and may also be attached to the absorbent assembly in the front waist seam 62 and the back waist seam 64. As such, the suspension members 102 and 104 may also assist in maintaining the positioning of the absorbent body 44 while the absorbent assembly 32 is subjected to forces in the longitudinal direction.

Alternatively, in another aspect representatively illustrated in FIG. 4, the training pants 20 of the present invention may include at least one suspension member 100 attached to each of the absorbent side edges 92 of the absorbent body 44 in each of the front and back waist regions 22, 24. In a particular aspect, there may be a plurality of suspension members 100 attached to each of the absorbent side edges 92 of the absorbent body 44 in each of the waist regions 22, 24. The suspension members 100 may extend from the absorbent side edges 92 of the absorbent body 44 and be attached to the absorbent assembly 32 in at least the waist seams 62, 64. For example, at least one suspension member 100 may extend from each of the absorbent side edges 92 of the absorbent body 44 in the front waist region 22 and may be attached to the absorbent assembly 32 in the front waist seams 62. Specifically, the suspension members 100 may be sandwiched between the outercover 40 and the bodyside liner 42 in the front waist seam 62. Similarly, at least one suspension member 100 may extend from each of the absorbent side edges 92 in the back waist region 24 and may be attached to the absorbent assembly 32 in the back waist seam 64. In particular, the suspension members 100 may be sandwiched between the outercover 40 and the bodyside liner 42 in the back waist seam 64. As such, the absorbent body 44 may be configured to float between the outercover 40 and the bodyside liner 42. Further, in such an arrangement, the suspension members 100 can assist in maintaining the positioning of the absorbent body 44 while the absorbent assembly 32 is subjected to forces in the lateral direction 49 and the longitudinal direction 48. Further, as illustrated in FIG. 4, additional suspension members 100 may be attached to the absorbent side edges 92 of the absorbent body 44 in the waist regions 22, 24 to be attached to the absorbent assembly 32 in one of the seams 61, 62, 64. In the aspect illustrated in FIG. 4, certain suspension members 100 are attached to the absorbent assembly in the side seams 61. Such a configuration can further aid in maintaining the positioning of the absorbent body 44, particularly when the assembly 32 is subjected to forces in the lateral direction 49.

Moreover, as mentioned above, the suspension members 100 may also be extensible, or more suitably elastomeric, for example in configurations where the outercover 40 and liner 42 are stretchable, or in particular aspects where the outercover 40 and liner 42 are elastomeric. In such an arrangement, the lateral extension of outercover 40 and liner 42 the waist regions 22 and 24 (such as during the donning of the article or during the course of wear of the article) may be less inhibited by the stretchable nature of the suspension members 100. As such, the fit and comfort of the article may be further improved by the suspension members 100.

Likewise, following the insult of the training pants 20, the absorbent body 44 may have a tendency to sag or droop in the crotch region 26. As mentioned above, the suspension members 100 can aid in maintaining the positioning of the absorbent body 44 relative to the outercover 40 when subjected to such loading. In particular, where the outercover 40 and the liner 42 are stretchable, the sag of the absorbent body 44 may cause components of the article (such as the containment flaps 46) to pull away from the wearer, compromising their performance. The suspension members 100 can assist in reducing the sag of the absorbent body 44 in the longitudinal direction 49, and in particular, where the suspension members 100 are elastomeric, can allow the absorbent assembly 32 to conform to the wearer while still providing support to the absorbent body 44 in the longitudinal direction.

As may be appreciated by one of skill in the art, a multiple suspension member configuration (as illustrated in FIG. 4) may be used in one waist region in combination with a unitary suspension member configuration (illustrated in FIG. 3 and 7) in the opposing waist region. Moreover, suspension members 100 may also be advantageously located in the crotch region of the article for greater support of the absorbent body. Such an arrangement may be beneficial where the absorbent may be subjected to particularly heavy loading (for example, in an overnight product) or in a situation where the training pants are configured for a very active user.

In the illustrated aspects, the suspension members 100 are generally rectilinear, and more specifically rectangular. Nonetheless, the suspension members 100 may be of various suitable shapes as are well known in the art. For example, the suspension members 100 may be generally circular, curvilinear, oval, triangular, and the like or combinations thereof. Likewise, the suspension members 100 of the various aspects of the present invention may be of various sizes. For example, as representatively illustrated in FIGS. 3 and 7, the suspension members 100 each define a width in the lateral direction 49 that is generally equal to the width in the lateral direction 49 of the absorbent assembly 32. Alternatively, the suspension members 100 may each define a width in the lateral direction 49 that is between 2% and 85% of the width of the absorbent assembly 32. Similarly, the suspension members 100 may each define a length in the longitudinal direction 48 that is between 5% and 25% of the length of the absorbent assembly 32 in the longitudinal direction 48.

Moreover, in particular aspects, at least a portion of each of the suspension members 100 may be in alignment with at least a portion of the absorbent body 44. That is, as representatively illustrated in FIGS. 3 and 7, at least a portion of each of the suspension members 100 may be overlapped, or overlap at least a portion of the absorbent body 44 in the longitudinal direction 48. In particular, in one aspect, at least a portion of each of the suspension members 100 may be in alignment with at least 5% of the absorbent body 44 in the longitudinal direction 48. More particularly, at least a portion of each of the suspension members 100 may be in alignment with at least 15% of the absorbent body 44 in the longitudinal direction 48.

The suspension members 100 may be formed from a wide variety of materials. Further, as mentioned above, the suspension members 100 may be stretchable, and still further, elastomeric. The members 100 may, for example, be composed of a single layer, multiple layers, laminates, spunbond fabrics, films, meltblown fabrics, elastic netting, microporous web, bonded carded webs or foams provided by elastomeric or polymeric materials. Elastomeric nonwoven laminate webs may include a nonwoven material joined to one or more gatherable nonwoven webs, films, or foams. Stretch Bonded Laminates (SBL) and Neck Bonded Laminates (NBL) are examples of elastomeric composites. Nonwoven fabrics are any web of material which has been formed without the use of textile weaving processes which produce a structure of individual fibers which are interwoven in an identifiable repeating manner. Examples of suitable materials are Spunbond-Meltblown fabrics, Spunbond-Meltblown-Spunbond fabrics, Spunbond fabrics, or laminates of such fabrics with films, foams, elastic strand/non-woven laminates, or other nonwoven webs. Elastomeric materials may include cast or blown films, foams, or meltblown fabrics composed of polyethylene, polypropylene, or polyolefin copolymers, as well as combinations thereof. The suspension members 100 may include materials that have elastomeric properties through a mechanical process, printing process, heating process, or chemical treatment. For example such materials may be apertured, creped, neck-stretched, heat activated, embossed, and micro-strained; and may be in the form of films, webs, and laminates.

In stretchable configurations, the suspension members 100 can suitably be stretched, laterally and/or longitudinally, by at least 30% (to at least 130% of an initial (unstretched) width and/or length of the suspension member 100). More suitably, the suspension members 100 can be stretched laterally and/or longitudinally, by at least 50% (to at least 150% of the unstretched width or length of the suspension member 100). Even more suitably, the liner 42 can be stretched, laterally and/or longitudinally, by at least 100% (to at least 200% of the unstretched width or length of the suspension member 100). Tension force in the suspension member 100 at 50% extension is suitably between 50 and 1000 grams, more suitably between 100 and 600 grams, as measured on a 3 inch (7.62 cm) wide piece of the suspension member material.

In determining the extensibility or elastomeric nature in a particular of certain materials described herein, such as the outer cover materials, liner materials, suspension member materials, and absorbent composites, a sample may be taken from a manufactured web or from a finished product.

Where a sample is prepared from a manufactured web (prior to its incorporation in a product), specimens should be obtained from a segment of the web with consistent and even formation, such as along the midline of the web. Where the desired materials cannot be obtained from a manufactured web, the sample may be extracted from within the product. Care should be taken to avoid stretching layers during separation. The sample to be separated should be cut to the desired specimen dimensions, or, depending on adhesive chemistry, the sample section may be treated with a solvent selected to dissolve a binding adhesive without affecting the structure or properties of the constituent layers. Each specimen to be tested should be free from attachment to any other components that may be present, such as leg or waist elastic structures, etc., at least in the region to be tested.

Where a given material or product will not permit specimens of the desired dimensions (for example, 3 inches (7.62 cm) by 3 inches (7.62 cm)) to be prepared, the material selected should be as long as possible to allow sufficient material at the ends of the sample (i.e. at least ½ inch/13 mm) for gripping.

All specimens of a given sample should be tested at the same dimensions. Where the dimensions for a set of specimens is under 2 inches (5.08 cm), at least 10 specimens of each sample should be tested, and their results used in determining the average value for that sample.

When introducing elements of the present invention or the preferred aspect(s) thereof, the articles “a”, “an”, “the”, and “said” are intended to mean that there are one or more of the elements. The terms “comprising,” “including”, and “having” are intended to be inclusive and mean that there may be additional elements other than the listed elements.

As various changes could be made in the above constructions without departing from the scope of the invention, it is intended that all matter contained in the above description or illustrated in the accompanying drawings shall be interpreted as illustrative and not in a limiting sense.

1. An absorbent article defining a front waist region, a back waist region, a crotch region that extends between and connects said front and back waist regions, a longitudinal direction and a lateral direction, an inner surface, an outer surface opposite said inner surface, and a pair of longitudinally opposed end margins and a pair of laterally opposed side margins, said article comprising:
A stretchable outercover;
A stretchable bodyside liner joined to the stretchable outercover in a superposed relation wherein said outercover and said bodyside liner are joined along at least a portion of each of said end margins to provide a front waist seam and a back waist seam and wherein said outercover and said bodyside liner are joined along at least a portion of each of said side margins in said front waist region and said back waist region to provide a pair of side seams in each of said front waist region and said back waist region;
An absorbent body disposed between said outercover and said bodyside liner, said absorbent body defining a pair of longitudinally opposed absorbent end edges and a pair of laterally opposed absorbent side edges;
A first elastomeric suspension member attached to said absorbent body in said front waist region; and
A second elastomeric suspension member attached to said absorbent body in said back waist region;
Wherein said first elastomeric suspension member is sandwiched between said outercover and said bodyside liner in said pair of side seams in said front waist region, and wherein said second elastomeric suspension member is sandwiched between said outercover and said bodyside liner in said pair of side seams in said back waist region.

2. The absorbent article of claim 1 wherein said first suspension member defines a first suspension member width in said lateral direction that is substantially equal to an article width in said front waist region and said second suspension member defines a second suspension member width in said lateral direction that is substantially equal to an article width in said back waist region.

3. The absorbent article of claim 1 wherein said first suspension member is sandwiched between said outercover and said bodyside liner in said front waist seam.

4. The absorbent article of claim 1 wherein said second suspension member is sandwiched between said outercover and said bodyside liner in said back waist seam.

5. The absorbent article of claim 1 wherein each of said suspension members is biaxially stretchable.

6. The absorbent article of claim 1 wherein each of said suspension members may be elongated in at least one direction at least 30%.

7. The absorbent article of claim 1 wherein each of said suspension member may be elongated in at least one direction at least 50%.

8. The absorbent article of claim 1 wherein at least a portion of each of said suspension members is in alignment with at least 5% of said absorbent body in the longitudinal direction.

9. The absorbent article of claim 1 wherein said absorbent body is extensible.

10. The absorbent article of claim 1 wherein said absorbent body is elastomeric.

11. The absorbent article of claim 1 wherein said absorbent side edges are non-parallel and said absorbent body includes a pair of lateral extensions in said front waist region and a pair of lateral extensions in said back waist region.

12. The absorbent article of claim 1 wherein said outercover is elastomeric.

13. The absorbent article of claim 1 wherein said outercover may be elongated at least 50%.

14. The absorbent article of claim 1 wherein said bodyside liner is elastomeric.

15. The absorbent article of claim 1 wherein said bodyside liner may be elongated at least 50%.

16. The absorbent article of claim 1 wherein said absorbent body is configured to float between said outercover and said bodyside liner.

17. The absorbent article of claim 1 wherein said absorbent body is disposed toward said inner surface relative to said first and second suspension members.

18. A disposable absorbent article defining a front waist region, a back waist region, a crotch region that extends between and connects said front and back waist regions, a longitudinal direction and a lateral direction, said article comprising:
A stretchable absorbent assembly defining a pair of longitudinally opposed end margins and a pair of laterally opposed side margins, said stretchable absorbent assembly comprising:
i) A stretchable outercover;
ii) A stretchable bodyside liner joined to the stretchable outercover in a superposed relation to form a pair of absorbent assembly side seams and a pair of absorbent assembly end seams;
iii) A first elastomeric suspension member attached to said absorbent assembly in said front waist region;
iv) A second elastomeric suspension member attached to said absorbent assembly in said back waist region; and
v) An absorbent body disposed between said outercover and said bodyside liner;
Wherein said absorbent body is joined to said first elastomeric suspension member and said second elastomeric suspension member, and wherein said absorbent body is configured to float between said outercover and said bodyside liner.

19. The absorbent article of claim 18 wherein each of said suspension members is biaxially stretchable.

20. The absorbent article of claim 18 wherein each of said suspension member may be elongated in at least one direction at least 50%.

21. The absorbent article of claim 18 wherein said absorbent body is elastomeric.

22. The absorbent article of claim 18 wherein said outercover is elastomeric.

23. The absorbent article of claim 18 wherein said outercover may be elongated at least 50%.

24. The absorbent article of claim 18 wherein said bodyside liner is elastomeric.

25. The absorbent article of claim 18 wherein said bodyside liner may be elongated at least 50%.

26. A disposable absorbent article defining a front waist region, a back waist region, a crotch region that extends between and connects said front and back waist regions, a longitudinal direction and a lateral direction, and a pair of longitudinally opposed end margins and a pair of laterally opposed side margins, said article comprising:
A stretchable outercover;
A stretchable bodyside liner joined to the stretchable outercover in a superposed relation wherein said outercover and said bodyside liner are joined along at least a portion of said end margins in said front waist region and said back waist region to provide a front waist seam and a back waist seam and wherein said outercover and said bodyside liner are joined along at least a portion of said side margins in said front waist region and said back waist region to provide a pair of side seams in said front waist region and said back waist region;
An absorbent body disposed between said outercover and said bodyside liner, said absorbent body defining a pair of longitudinally opposed absorbent end edges and a pair of laterally opposed absorbent side edges;
At least one elastomeric suspension member attached to each of said absorbent side edges in said front waist region, wherein said at least one elastomeric suspension member is sandwiched between said outercover and said bodyside liner in said front waist seam; and
At least one elastomeric suspension member attached to each of said absorbent side edges in said rear waist region, wherein said at least one elastomeric suspension member is sandwiched between said outercover and said bodyside liner in said back waist seam.

27. The absorbent article of claim 26 wherein said absorbent body is configured to float between said outercover and said bodyside liner.

28. The absorbent article of claim 26 wherein each of said suspension members are biaxially stretchable.

29. The absorbent article of claim 26 wherein each of said suspension members may be elongated in at least one direction at least 50%.

30. The absorbent article of claim 26 wherein said absorbent body is elastomeric.

31. The absorbent article of claim 26 wherein said outercover may be elongated at least 50%.

32. The absorbent article of claim 26 wherein said bodyside liner is elastomeric.

33. The absorbent article of claim 26 wherein said bodyside liner may be elongated at least 50%.
